(19) United States
(12) Patent Application Publication
(10) Pub. No.: US 2005/0204485 A1
(43) Pub. Date: Sep. 22, 2005

(54) Composition for dyeing keratin fibers, containing an alcohol oxidase and an anionic associative polymer, process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,081

(22) Filed: Jan. 28, 2005

Related U.S. Application Data
(60) Provisional application No. 60/545,138, filed on Feb. 18, 2004.

(30) Foreign Application Priority Data
Jan. 28, 2004 (FR) 04 00778

(51) Int. Cl. A61K 7/13

(52) U.S. Cl. 8/406

(57) ABSTRACT

The present disclosure relates to a composition for dyeing keratin fibers, for instance human keratin fibers such as the hair, comprising, in a medium that is suitable for dyeing keratin fibers, at least one oxidation dye precursor, at least one alcohol oxidase enzyme, at least one substrate for the enzyme and at least one anionic associative polymer. The disclosure further relates to a process for dyeing keratin fibers, comprising applying the composition as disclosed, and also to a dyeing “kit”.

This application claims benefit of U.S. Provisional Application No. 60/545,138, filed Feb. 18, 2004, and of French Application FR 04/00778, filed Jan. 28, 2004, both of which are hereby incorporated by reference.

The present disclosure relates to a composition for dyeing keratin fibers, for instance human keratin fibers such as the hair, comprising, in a medium that is suitable for dyeing, at least one oxidation dye precursor, at least one alcohol oxidase enzyme, at least one substrate for the enzyme and at least one anionic associative polymer.

It is known practice to dye keratin fibers, for instance human hair, with dye compositions comprising oxidation dye precursors, such as ortho- or para-phenylenediamines, ortho- or para-aminophenols, and heterocyclic compounds, which are generally referred to as oxidation bases. These oxidation bases are colorless or weakly colored compounds which, when combined with oxidizing products, may give rise to colored compounds by a process of oxidative condensation.

It is also known that the shades obtained with these oxidation bases may be varied by combining them with couplers or coloration modifiers, wherein the coloration modifiers may be chosen from, for example, aromatic meta-diamines, meta-aminophenols, meta-diphenols and certain heterocyclic compounds such as indole compounds. The variety of molecules that may be used as oxidation bases and couplers makes it possible to obtain a wide range of colors.

The “permanent” coloration obtained by means of these oxidation dyes should, moreover, satisfy a certain number of requirements. Thus, it should have no toxicological drawbacks, should allow shades of the desired intensity to be obtained and/or should have good resistance to external agents (i.e., light, bad weather, washing, permanent waving, perspiration and rubbing). The dyes also ideally allow white hairs to be covered and are as unselective as possible, that is to say that they can allow the smallest possible differences in coloration to be produced over the entire length of the same keratin fiber, which is generally differently sensitized (i.e. damaged) between its end and its root.

Oxidation dyeing is generally performed in strongly alkaline medium, in the presence of hydrogen peroxide. However, the use of alkaline media in the presence of hydrogen peroxide can have the drawback of causing considerable degradation of the fibers, and also bleaching of keratin fibers, which is not always desirable.

The oxidation dyeing of keratin fibers may also be performed using oxidizing systems other than hydrogen peroxide, such as enzymatic systems. For example, French Patent Application No. FR 2 769 219 describes the use of a uricase enzyme and of its uric acid substrate in oxidation dyeing to dye keratin fibers. European Patent Application No. EP-A-0 310 675 describes the use of oxidation dye precursors of benzenic type in combination with enzymes such as pyranose oxidase and glucose oxidase. More recently, French Patent Application No. FR 2 833 492 describes the use of the alcohol oxidase enzyme as sole enzyme in an oxidation dye composition for dyeing keratin fibers.

Accordingly, one aspect of the present disclosure provides novel compositions for dyeing keratin fibers by oxidation dyeing, using an oxidizing system other than hydrogen peroxide.

As used herein, the term “thickened composition” is understood to mean a composition that can be applied to the area to be dyed without running outside this area.

For example, the viscosity of the compositions according to the present disclosure is greater than 200 cp, such as greater than 500 cp, measured at 25° C. using a Rheomat RM 180 rheometer at a shear rate of 200 s⁻¹.

Further, another aspect of the present disclosure relates to a composition comprising at least one oxidation dye precursor, at least one alcohol oxidase enzyme, at least one enzyme substrate for the enzyme and at least one anionic associative polymer, which can be used for dyeing keratin fibers, for instance human keratin fibers such as the hair, without the need for hydrogen peroxide.

The compositions according to the present disclosure make it possible to obtain homogeneous formulations which, once applied, can respect the nature of the keratin fibers, and may not have the solubilization and crystallization problems encountered, for example, with the uric acid/uricase system. The inventors have also noted that the stability of the compositions as disclosed herein, for instance, the stability of the alcohol oxidase enzyme of these compositions, is improved.

The compositions according to the present disclosure have the benefit that they can lead to the production of dyeing results with strong, unselective and fast colors, and these compositions are capable of generating varied shades of intense and uniform color, without any significant degradation of the hair. In addition, it has been noted that the use of such compositions can improve the hold of permanent-waved hair and reduce the porosity of the hair.

The present inventors have also observed that during the use of the composition as disclosed herein, the uptake of the dye onto the fibers is advantageously not halted. In contrast, the use of dye compositions containing conventional thickeners, surfactants and solvents generally does result in halting of the uptake of the dye. When the uptake of the dye onto the fibers is halted, producing a strong shade requires the use of a larger amount of dye and also a larger amount of solvent and/or of surfactant to dissolve this dye.

Other characteristics, aspects, subjects and benefits of the present disclosure will emerge more clearly upon reading the description and the examples that follow.

The anionic associative polymers that may be used in the compositions according to the present disclosure are water-soluble anionic polymers capable, in aqueous medium, of reversibly associating with each other or with other molecules. The at least one anionic associative polymer can be chosen from, for example, anionic amphiphilic polymers comprising at least one hydrophilic unit and at least one fatty-chain allyl ether unit and anionic amphiphilic polymers comprising at least one hydrophilic unit of olefinic unsaturated carboxylic acid type and at least one hydrophobic unit of the type such as a (C₁₀-C₃₀)alkyl ester of an unsaturated carboxylic acid.

The anionic amphiphilic polymers comprising at least one hydrophilic unit, and at least one fatty-chain allyl ether unit, used as disclosed herein may be chosen from, for instance, those whose hydrophilic unit comprises an ethylenic unsaturated anionic monomer, for example a vinylcarboxylic acid and such as an acrylic acid or a methacrylic acid, or mixtures thereof. The fatty-chain allyl ether unit of these anionic amphiphilic polymers can be chosen from the monomers of formula (I):
CH₂═CR′CH₂OBₙR (I)
wherein R′ is chosen from a hydrogen atom and CH₃ radicals, B is an ethyleneoxy radical, n is an integer ranging from 0 to 100, R is chosen from hydrocarbon-based radicals chosen from alkyl, arylalkyl, aryl, alkylaryl and cycloalkyl radicals comprising from 8 to 30 carbon atoms, for instance from 10 to 24, such as from 12 to 18 carbon atoms; for example, R may be chosen from alkyl and alkylaryl hydrocarbon-based radicals comprising from 10 to 24 carbon atoms.

In one embodiment of the present disclosure, R′ is a hydrogen atom, n is equal to 10, and R is a stearyl (C18) radical.

For example, anionic amphiphilic polymers of this type are described and prepared, according to an emulsion polymerization process, in European Patent No. EP-0 216 479 B2.

Among these anionic amphiphilic polymers formed by emulsion polymerization that may be used according to the present disclosure, non-limiting mention may be made of polymers formed from 20% to 60% by weight of acrylic acid and/or of methacrylic acid, from 5% to 60% by weight of lower alkyl (meth)acrylates, from 2% to 50% by weight of fatty-chain allyl ether of formula (I), and from 0% to 1% by weight of a crosslinking agent which is conventionally a well-known copolymerizable unsaturated polyethylenic monomer, for instance diallyl phthalate, allyl (meth)acrylate, divinylbenzene, (poly)ethylene glycol dimethacrylate or methylenebisacrylamide. Among the latter polymers, further non-limiting mention may be made of crosslinked terpolymers of methacrylic acid, of ethyl acrylate and of polyethylene glycol (10 EO) stearyl ether (Steareth-10), for instance those sold by the company Allied Colloids under the names Salcare SC 80 and Salcare SC 90, which are aqueous 30% emulsions of a crosslinked terpolymer of methacrylic acid, of ethyl acrylate and of steareth-10 allyl ether (40/50/10).

The anionic amphiphilic polymers comprising at least one hydrophilic unit of olefinic unsaturated carboxylic acid type and at least one hydrophobic unit of the type such as a (C₁₀-C₃₀)alkyl ester of an unsaturated carboxylic acid, which are used according to the present disclosure can be chosen from, for instance, those whose hydrophilic unit of olefinic unsaturated carboxylic acid type is chosen from the monomers of formula (II):
CH₂═CR₁COOH (II)
wherein R₁ is chosen from a hydrogen atom, and CH₃ and C₂H₅ radicals, i.e. acrylic acid, methacrylic acid or ethacrylic acid units. For example, the polymer used can be such that the hydrophilic unit of olefinic unsaturated carboxylic acid type is an acrylic acid or methacrylic unit, or mixtures thereof.

The hydrophobic unit of the type such as a (C₁₀-C₃₀)alkyl ester of an unsaturated carboxylic acid of the anionic amphiphilic polymer can be chosen from the monomers of formula (III):
CH₂═CR₁COOR₂ (III)
wherein R₁ is chosen from a hydrogen atom, and CH₃ and C₂H₅ radicals (i.e. acrylate, methacrylate or ethacrylate units), such as acrylate units or methacrylate units, R₂ is chosen from C₁₀-C₃₀, such as, C₁₂-C₂₂, alkyl radicals.

In one embodiment of the present disclosure, the anionic amphiphilic polymer is crosslinked.

Non-limiting examples of (C₁₀-C₃₀)alkyl esters of unsaturated carboxylic acids that can be used in accordance with the present disclosure include, for instance, lauryl acrylate, stearyl acrylate, decyl acrylate, isodecyl acrylate and dodecyl acrylate and the corresponding methacrylates, lauryl methacrylate, stearyl methacrylate, decyl methacrylate, isodecyl methacrylate and dodecyl methacrylate.

Anionic amphiphilic polymers of this type can be disclosed and prepared, for example, according to U.S. Pat. Nos. 3,915,921 and 4,509,949.

Among the anionic amphiphilic polymers of this type that can be used in the context of the present disclosure, non-limiting mention may be made of polymers formed from a mixture of monomers comprising:

(i) essentially acrylic acid, an ester of formula:
CH₂═CR₁COOR₂
wherein R₁ is chosen from a hydrogen atom and CH₃ radicals, R₂ is chosen from alkyl radicals comprising from 12 to 22 carbon atoms, and a crosslinking agent, such as, for example, those comprising from 95% to 60% by weight of acrylic acid (hydrophilic unit), 4% to 40% by weight of C₁₀-C₃₀ alkyl acrylate (hydrophobic unit), and 0% to 6% by weight of a crosslinking polymerizable monomer, or 98% to 96% by weight of acrylic acid (hydrophilic unit), 1% to 4% by weight of C₁₀-C₃₀ alkyl acrylate (hydrophobic unit) and 0.1% to 0.6% by weight of a crosslinking polymerizable monomer,
(ii) essentially acrylic acid and lauryl methacrylate, such as the product formed from 66% by weight of acrylic acid and 34% by weight of lauryl methacrylate.

The crosslinking agent can be a monomer comprising a group 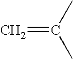
with at least one other polymerizable group whose unsaturated bonds are not conjugated. Non-limiting mention may be made, for example, of polyallyl ethers such as, polyallylsucrose and polyallylpentaerythritol.

Among the polymers above, further non-limiting mention may be made of the products sold by the company Goodrich under the trade names Pemulen TR1, Pemulen TR2, Carbopol 1382, and the product sold by the company S.E.P.C. under the name Coatex SX. In one embodiment of the present disclosure, Pemulen TR1 is used.

The at least one anionic amphiphilic polymer can be present in the compositions as disclosed herein, for example, in an amount ranging from 0.01% to 10% by weight, such as, from 0.1% to 5% by weight, relative to the total weight of the dye composition applied to the fibers.

In the context of the present disclosure, the at least one alcohol oxidase enzyme used in the dye compositions as disclosed herein belongs to the class E.C.1.1.3 of the enzyme nomenclature (see Enzyme Nomenclature, Academic Press Inc; 1992).

Thus, the at least one alcohol oxidase enzyme can be chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

In one embodiment of the present disclosure, the at least one alcohol oxidase enzyme used in the composition is a primary alcohol oxidase (EC 1.1.3.13).

Alcohol oxidase enzymes form a particular class of 2-electron oxidoreductase enzymes. The at least one alcohol oxidase enzyme used in the dye composition as disclosed herein can be derived from an extract of plants, of animals, of micro-organisms (bacterium, fungus, yeast, microalga or virus), of differentiated or undifferentiated cells, obtained in vivo or in vitro, unmodified or genetically modified, or synthetic (obtained by chemical or biotechnological synthesis).

By way of non-limiting example, the at least one alcohol oxidase enzyme may be derived from the species: Rhodococcus erythropolis, Pseudomonas pseudoalcaligenes which are bacteria, Aspergillus niger, Kamagataella pastoris, Phanerochaete chrysosporium, Polyporus obtusus, Hansenula polymorpha, Poria contigua, Penicillium simplicissimum, Pleurotus pulmonarius (fungi), Pichia sp. (pastoris, methanolica, angusta) and Candida sp. (boidinii, albicans, tropicalis) (yeasts), Pinus strobus, which is a species of plant origin, Gastropode mollusc and Manduca sexta, which are of animal origin.

In one embodiment of the present disclosure, the at least one alcohol oxidase enzyme used in the composition is an alcohol oxidase derived from Pichia pastoris.

For example, the at least one alcohol oxidase enzyme can be present the dye composition in an amount ranging from 0.05% to 20% by weight, relative to the total weight of the composition, for instance, from 0.1% to 10% by weight, and such as from 0.5% to 8% by weight, relative to the weight of the composition.

The enzymatic activity of the alcohol oxidase enzymes used in accordance with the present disclosure may be defined from the oxidation of the donor under aerobic conditions. One unit U corresponds to the amount of enzyme leading to the generation of 1 μmol of hydrogen peroxide per minute at a given pH and at a temperature of 25° C.

The at least one alcohol oxidase enzyme can be present, for example, in an amount ranging from 10³ U to 10⁵ U, such as from 2×10³ U to 5×10⁴ U, per 100 g of dye composition.

The at least one substrate for the enzyme is also known as a donor for the enzyme. The at least one substrate for the enzyme as disclosed herein can be, for instance, an alcohol chosen from primary alcohols, secondary alcohols, long-hydrocarbon-chain alcohols and aromatic alcohols. For example, among the donors for the primary alcohol oxidases that may be used, non-limiting mention can be made of primary alcohols comprising from 1 to 6 carbon atoms; among the donors for the aryl alcohol oxidases that may be used, non-limiting mention may be made of benzyl alcohol, 4-tert-butylbenzyl alcohol, 3-hydroxy-4-methoxybenzyl alcohol, veratryl alcohol, 4-methoxybenzyl alcohol and cinnamyl alcohol. In addition, 2,4-hexadien-1-ol may also be used as donor for the aryl alcohol oxidases.

According to another aspect of the present disclosure, the substrate for the enzyme is a compound bearing at least one aliphatic or aromatic alcohol functional group, suitable for reacting with the enzyme used. The compound bearing at least one aliphatic or aromatic alcohol functional group may be, for instance, an oxidation dye precursor or a cosmetically acceptable adjuvant, for example a polymer, a surfactant or a preserving agent bearing at least one alcohol functional group. For further instance, the substrate for the enzyme can be an oxidation dye precursor bearing at least one aliphatic or aromatic alcohol functional group. For example, N-(β-hydroxypropyl)-para-phenylenediamine, which bears a primary alcohol functional group, may serve as oxidation base and as substrate for the alcohol oxidase. Similarly, oxidation couplers, such as meta- or para-aminophenol, may fulfil both functions. Such precursors are described hereinbelow. In this instance, the use of other substrates for the enzyme is optional.

Thus, the present disclosure relates to a composition for dyeing keratin fibers, for instance human keratin fibers such as the hair, comprising, in a medium that is suitable for dyeing, at least one oxidation dye precursor; at least one alcohol oxidase enzyme; at least one substrate bearing an alcohol functional group for the enzyme, and at least one anionic associative polymer, the said substrate possibly being replaced, totally or partially, with the oxidation dye precursor in the case where the oxidation dye precursor bears at least one aliphatic or aromatic alcohol functional group.

The use of the composition as disclosed herein can make it possible to reduce the risks associated with handling of hydrogen peroxide. Furthermore, the concentration of preserving agents in the compositions according to the present disclosure may be reduced by providing compounds comprising an alcohol functional group that also have preserving properties.

The at least one substrate for the enzyme can be present in an amount, for example, ranging from 0.01% to 60% by weight, relative to the total weight of the composition, such as ranging from 0.05% to 30% by weight, relative to the total weight of the composition.

The at least one oxidation base may be chosen from, for example, para-phenylenediamines, bis(phenyl)alkylenediamines, para-aminophenols, ortho-aminophenols, heterocyclic bases, and the addition salts thereof.

Among the para-phenylenediamines, non-limiting mention may be made, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxy-ethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-paraphenylenediamine, N,N-(ethyl-β-hydroxyethyl)-para-phenylenediamine, N-(βγ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, further non-limiting mention can be made of para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the addition salts thereof with an acid are most particularly preferred.

Among the bis(phenyl)alkylenediamines, non-limiting mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols, non-limiting mention may be made, by way of example, of para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethyl phenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol and bis(5′-amino-2′-hydroxy)phenylmethane and the addition salts thereof with an acid.

Among the ortho-aminophenols, non-limiting mention may be made, by way of example, of 2-aminophenol, 2-amino-5-methylphenol, 2-amino-6-methylphenol and 5-acetamido-2-aminophenol, and the addition salts thereof with an acid.

Among the heterocyclic bases, non-limiting mention may be made, by way of example, of pyridine derivatives, pyrimidine derivatives and pyrazole derivatives.

Among the pyridine derivatives, non-limiting mention may be made of the compounds described, for example, in British Patent Nos. GB 1 026 978 and GB 1 153 196, as well as 2,5-diaminopyridine, 2-(4-methoxyphenyl)amino-3-aminopyridine, and 3,4-diaminopyridine, and the addition salts thereof with an acid.

Other pyridine oxidation bases that can be useful in the present disclosure include the 3-aminopyrazolo[1,5-a]pyridine oxidation bases or the addition salts thereof described, for example, in French Patent Application No. FR 2 801 308. By way of non-limiting example, mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminnopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazoio[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol;
and also the addition salts thereof with an acid.

Among the pyrimidine derivatives, non-limiting mention may be made of the compounds described, for example, in German Patent No. DE 2 359 399; Japanese Patent Nos. JP 88-169 571 and JP 05 163 124; European Patent No. EP 0 770 375 or International Patent Application No. WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine and the addition salts thereof and the tautomeric forms thereof, when a tautomeric equilibrium exists.

Among the pyrazole derivatives, non-limiting mention may be made of the compounds described in German Patent Nos. DE 3 843 892 and DE 4 133 957, and International Patent Application Nos. WO 94/08969 and WO 94/08970, French Patent Application No. FR-A-2,733,749 and German Patent Application No. DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-4-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof.

The at least one oxidation base can be present in an amount, for example, ranging from 0.0001% to 20%, such as 0.005% to 6%, by weight relative to the total weight of the composition.

Among standard oxidation couplers that may be used as disclosed herein, non-limiting mention may be made of meta-phenylenediamines, meta-aminophenols, meta-diphenols, naphthalenic couplers, heterocyclic couplers, and also the addition salts thereof.

Further non-limiting examples that may be mentioned include 2-methyl-5-aminophenol, 5-N-(β-hydroxyethyl)amino-2-methyl phenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

The at least one oxidation coupler can be present in an amount, for example, ranging from 0.0001% to 20%, such as from 0.005% to 6%, by weight relative to the total weight of the composition.

The acid addition salts that may be used for the oxidation bases and couplers may be chosen from, for example, the hydrochlorides, hydrobromides, sulphates, citrates, succinates, tartrates, lactates, tosylates, benzenesulphonates, phosphates and acetates.

The base addition salts that may be used as disclosed herein may be chosen, for example, from the addition salts with sodium hydroxide, potassium hydroxide, ammonia, amines and alkanolamines.

The dye composition as disclosed herein may also comprise at least one direct dye, which may be chosen for example, from neutral, acidic or cationic nitrobenzene dyes, neutral, acidic or cationic azo direct dyes, neutral, acidic or cationic quinone and, for instance, anthraquinone direct dyes, azine direct dyes, methine, azomethine, triarylmethane and indoamine direct dyes, and natural direct dyes. In one embodiment of the present disclosure, the at least one direct dye is chosen from cationic direct dyes and natural direct dyes.

Among the cationic direct dyes that may be used according to the present disclosure, non-limiting mention may be made of the cationic azo direct dyes described in International Patent Application Nos. WO 95/15144 and WO 95/01772, and European Patent Application No. EP 714 954.

Among these compounds, further non-limiting mention may be made of the following dyes:
1,3-dimethyl-2-[[4-(dimethylamino)phenyl]azo]-1H-imidazolium chloride
1,3-dimethyl-2-[(4-aminophenyl)azo]-1H-imidazolium chloride, and
1-methyl-4-[(methylphenylhydrazino)methyl]pyridinium methyl sulphate.

Among the natural direct dyes that may be used according to the present disclosure, non-limiting mention may be made of lawsone, juglone, alizarin, purpurin, carminic acid, kermesic acid, purpurogallin, protcatechaldehyde, indigo, isatin, curcumin, spinulosin and apigenidin. Extracts or decoctions comprising these natural dyes, such as henna-based poultices or extracts, may also be used.

The at least one direct dye can be present in the composition in an amount, for example, ranging from 0.001% to 20% by weight, for instance, from 0.005% to 10% by weight, relative to the total weight of the ready-to-use composition.

The dye composition in accordance with the present disclosure may also contain at least one adjuvant conventionally used in compositions for dyeing the hair, such as antioxidants, penetrating agents, sequestering agents, fragrances, buffers, dispersants, surfactants, conditioners such as, for example, volatile or non-volatile, modified or unmodified silicones, cationic polymers, cations, film-forming agents, ceramides, preserving agents, opacifiers and vitamins or provitamins.

The at least one adjuvant can be present in an individual amount ranging from 0.01% to 20% by weight, relative to the weight of the composition.

Needless to say, the person skilled in the art will take care to select this or these optional additional compound(s) such that the beneficial properties intrinsically associated with the oxidation dye composition in accordance with the present disclosure are not, or are not substantially, adversely affected by the envisaged addition(s).

The medium that is suitable for dyeing, also known as the dye support, can consists of water, or comprise a mixture of water and at least one organic solvent to dissolve the compounds that would not be sufficiently water-soluble. As appropriate, this solvent may be a substrate of the enzyme, such as ethanol or isopropanol. It may also be a non-substrate compound of the enzyme such as polyol ethers, for instance 2-butoxyethanol, propylene glycol, propylene glycol monomethyl ether and diethylene glycol monomethyl ether and monoethyl ether, or phenoxyethanol, and mixtures thereof.

The at least one organic solvent can be present in an amount, for example, ranging from 1% to 40% by weight, relative to the total weight of the dye composition, such as from 5% to 30% by weight.

The pH of the dye composition in accordance with the present disclosure can range from 6 to 11, for instance, from 7 to 10. It may be adjusted for the desired value using acidifying or basifying agents usually used in the dyeing of keratin fibers, or alternatively using standard buffer systems.

Among the acidifying agents that may be used, non-limiting mention may be made of, for example, mineral or organic acids, for instance hydrochloric acid, orthophosphoric acid, sulphuric acid, carboxylic acids, for instance acetic acid, tartaric acid, citric acid and lactic acid, and sulphonic acids.

Among the basifying agents that may be used, non-limiting mention may be made of for example, aqueous ammonia, alkyl metal carbonates, alkanolamines such as monoethanolamine, diethanolamine and triethanolamine and also derivatives thereof, sodium hydroxide, potassium hydroxide and the compounds of formula (III): 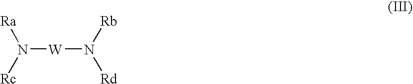
wherein W is a propylene residue optionally substituted with a hydroxyl group or a C₁-C₄ alkyl radical; Ra, Rb, Rc and Rd, which may be identical or different, are chosen from hydrogen atoms and C₁-C₄ alkyl and C₁-C₄ hydroxyalkyl radicals.

The dye composition may be in various forms, such as in the form of thickened liquids, creams or gels, or in any other form that is suitable for dyeing keratin fibers, and such as human hair.

When the at least one oxidation dye and the at least one alcohol oxidase are present in the same ready-to-use composition, the composition can be, for example, free of oxygen gas, so as to avoid any premature oxidation of the at least one oxidation dye.

Another aspect of the present disclosure is a process for dyeing keratin fibers, for instance human keratin fibers such as the hair, wherein the at least one dye composition according to the present disclosure is applied to the fibers, the duration of this application being a period of time that is sufficient to develop the desired coloration. The color is then revealed by bringing the alcohol oxidase enzyme and its substrate into contact in the presence of oxygen.

The composition is applied to the keratin fibers. After leaving it to act for a period of time ranging from 3 to 60 minutes, such as from 5 to 40 minutes, the keratin fibers are rinsed, washed with shampoo, rinsed again and then dried.

When the dye composition is a composition in ready-to-use form, it comprises, in a medium that is suitable for dyeing keratin fibers, at least one oxidation dye precursor, at least one alcohol oxidase enzyme, at least one substrate for the enzyme and at least one anionic associative polymer, and the mixture is then stored in anaerobic form, free of oxygen gas.

According to another aspect of the present disclosure, the process can comprise at least one composition (A) comprising in a medium that is suitable for dyeing keratin fibers at least one oxidation dye precursor and, at least one composition (B) comprising, in a medium that is suitable for dyeing keratin fibers, at least one alcohol oxidase enzyme, wherein the composition (A) and/or the composition (B) further comprise at least one substrate for the enzyme, and the composition (A) and/or the composition (B) further comprise at least one anionic associative polymer, wherein the compositions (A) and (B) are mixed together at the time of use, before applying the mixture to the keratin fibers.

According to yet another aspect of the present disclosure, the process comprises a composition (A) comprising in a medium that is suitable for dyeing keratin fibers at least one oxidation dye precursor, at least one substrate for the enzyme, and at least one anionic associative polymer and, a composition (B) comprising, in a medium that is suitable for dyeing keratin fibers, at least one alcohol oxidase enzyme, wherein the compositions (A) and (B) are mixed together at the time of use before applying the mixture to the keratin fibers.

The color may be revealed at acidic, neutral or alkaline pH. In the case where the process is performed using a composition (A) comprising at least one oxidation dye precursor, at least one substrate for the enzyme and at least one anionic associative polymer and a composition (B) comprising at least one alcohol oxidase enzyme, the enzyme may be added to the composition as disclosed herein just at the time of use, or it may be used starting with a composition comprising it, applied simultaneously or sequentially to the composition (A). This composition comprising the at least one alcohol oxidase enzyme, the so-called oxidizing composition may also comprise at least one adjuvant conventionally used in compositions for dyeing the hair and as defined above.

The pH of the composition (B), the so-called oxidizing composition, is such that, after mixing with the dye composition (A), the pH of the resultant composition applied to the keratin fibers will range from 6 to 11, such as from 7 to 10. It may be adjusted to the desired value by means of acidifying or basifying agents usually used in the dyeing of keratin fibers and as defined above.

For example, the application of the composition as disclosed herein can be carried out at a temperature ranging from the ambient temperature to 220° C., such as from the ambient temperature and 60° C.

Another aspect of the present disclosure is a multi-compartment device or dyeing “kit,” wherein at least one first compartment comprises the composition (A) as defined above and at least one second compartment comprises the composition (B) as defined above. This kit may be also be equipped with a tool for applying the desired mixture to the hair, such as the devices described in French Patent No. FR-2 586 913.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner.

### EXAMPLES

The following compositions were prepared:

| Constituents | Composition 1 | | Composition 2 | |
|---|---|---|---|---|
| Methacrylic acid/ethyl | 1 | g (AM) | | |
| acrylate/Steareth-10-allyl | | | | |
| ether crosslinked anionic | | | | |
| polymer sold as an aqueous | | | | |
| emulsion containing 30% AM | | | | |
| (Salcare SC 80 - Allied | | | | |
| Colloids) | | | | |
| Acrylic acid/C₁₀-C₃₀ alkyl | | | 1 | g (AM) |
| acrylate crosslinked anionic | | | | |
| amphiphilic polymer | | | | |
| (Pemulen TR16 - Goodrich) | | | | |
| Ethanol | 25 | g | 25 | g |
| para-Phenylenediamine | 3 × 10⁻³ | mol | 3 × 10⁻³ | mol |
| meta-Aminophenol | 3 × 10⁻³ | mol | 3 × 10⁻³ | mol |
| Alcohol oxidase | 20 000 | units U | 20 000 | units U |
| 2-Amino-2-methyl-1-propanol | qs pH 7 | | qs pH 7 | |
| Distilled water | qs 100 | g | qs 100 | g |

The alcohol oxidase used was the product sold by the company Biozyme Laboratories in liquid form at a concentration of 1980 units/ml. The unit U is the amount of enzyme leading to the generation of 1 μmol of hydrogen peroxide per minute at pH 7.5 (100 mM phosphate buffer) and at a temperature of 25° C.

The above compositions were applied to locks of natural grey permed hair comprising 90% white hairs, and left to act for 30 minutes. The bath ratio was set at 5. The alcohol oxidase was added extemporaneously. The hair was then rinsed, washed with a standard shampoo and then dried.

The hair was dyed in shades of khaki green.

1. A composition for dyeing keratin fibers, comprising, in a medium that is suitable for dyeing:
at least one oxidation dye precursor;
at least one alcohol oxidase enzyme;
at least one substrate bearing an alcohol functional group for the enzyme and
at least one anionic associative polymer,
wherein the at least one substrate may be totally or partially substituted by the at least one oxidation dye precursor when the at least one oxidation dye precursor bears at least one aromatic or aliphatic alcohol functional group.

2. The composition according to claim 1, wherein the at least one anionic associative polymer is chosen from anionic amphiphilic polymers comprising at least one hydrophilic unit and at least one fatty-chain allyl ether unit; and anionic amphiphilic polymers comprising at least one hydrophilic unit of olefinic unsaturated carboxylic acid type and at least one hydrophobic unit ester.

3. The composition according to claim 2, wherein the at least one hydrophobic unit is a (C₁₀-C₃₀)alkyl ester of an unsaturated carboxylic acid.

4. The composition according to claim 2, wherein the at least one anionic associative polymer is an anionic amphiphilic polymer comprising at least one hydrophilic unit and at least one fatty-chain allyl ether unit, wherein the at least one hydrophilic unit is an ethylenic unsaturated anionic monomer.

5. The composition according to claim 4, wherein the at least one hydrophilic unit is a vinylcarboxylic acid.

6. The composition according to claim 5, wherein the at least one hydrophilic unit is chosen from acrylic acid and methacrylic acid.

7. The composition according to claim 2, wherein the at least one fatty-chain allyl ether unit is chosen from monomers of formula (I):
CH₂═CR′CH₂OBₙR (I)
wherein R′ is chosen from a hydrogen atom and CH₃ radicals, B is an ethyleneoxy
radical, n is an integer ranging from 0 to 100, and R is chosen from alkyl, arylalkyl,
aryl, alkylaryl and cycloalkyl radicals comprising from 8 to 30 carbon atoms.

8. The composition according to claim 7, wherein R is chosen from alkyl and alkylaryl radicals comprising from 10 to 24 carbon atoms.

9. The composition according to claim 7, wherein R′ is a hydrogen atom, n is equal to 10 and R is a stearyl radical.

10. The composition according to claim 7, wherein the at least one anionic amphiphilic polymer is formed by emulsion polymerization from 20% to 60% by weight of acrylic acid and/or of methacrylic acid, from 5% to 60% by weight of lower alkyl (meth)acrylates, from 2% to 50% by weight of fatty-chain allyl ethers of formula (I), and from 0 to 1% by weight of a crosslinking agent.

11. The composition according to claim 10, wherein the at least one anionic amphiphilic polymer is a crosslinked terpolymer of methacrylic acid, of ethyl acrylate, and of polyethylene glycol (10 EO) stearyl ether.

12. The composition according to claim 1, wherein the at least one anionic amphiphilic polymer comprises at least one hydrophilic unit of olefinic unsaturated carboxylic acid type and at least one hydrophobic unit,
wherein the at least one hydrophilic unit of olefinic unsaturated carboxylic acid type is chosen from the monomers of formula (II):
CH₂═CR₁COOH (II)
wherein R₁ is chosen from a hydrogen atom and CH₃ C₂H₅ radicals.

13. The composition according to claim 12, wherein the at least one hydrophilic unit of olefinic unsaturated carboxylic acid type is chosen from acrylic acid and methacrylic acid.

14. The composition according to claim 12, wherein the at least one hydrophobic unit is chosen from monomers of formula (III):
CH₂═CR₁COOR₂ (III)
wherein R₁ is chosen from a hydrogen atom, and CH₃ and C₂H₅ radicals and R₂ is chosen from C₁₀-C₃₀ alkyl radicals.

15. The composition according to claim 14, wherein R₁ is chosen from a hydrogen atom and CH₃ radicals.

16. The composition according to claim 14, wherein R₂ is chosen from C₁₂-C₂₂ alkyl radicals.

17. The composition according to claim 12, wherein the at least one anionic amphiphilic polymer is crosslinked.

18. The composition according to claim 17, wherein the at least one anionic amphiphilic polymer is formed from a monomer mixture comprising acrylic acid, esters of formula
CH₂═CR₁COOR₂
wherein R₁ is chosen from a hydrogen atom and CH₃ radicals, and R₂ is chosen
from C₁₂-C₂₂ alkyl radicals,
and a crosslinking agent.

19. The composition according to claim 12, wherein the at least one anionic amphiphilic polymer is a polymer of acrylic acid and of lauryl methacrylate.

20. The composition according to claim 2, wherein the at least one anionic amphiphilic polymer is present in an amount ranging from 0.01% to 10% by weight, relative to the total weight of the composition.

21. The composition according to claim 1, wherein the at least one alcohol oxidase enzyme is chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

22. The composition according to claim 21, wherein the at least one alcohol oxidase enzyme is derived from a species chosen from: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp. (pastoris, methanolica, angusta), Candida sp. (boidinii, albicans, tropicalis), Pinus strobus, Gastropode mollusc, and Manduca sexta.

23. The composition according to claim 1, wherein the at least one alcohol oxidase enzyme is present in an amount ranging from 0.05% to 20% by weight, relative to the total weight of the composition.

24. The composition according to claim 1, wherein the the at least one alcohol functional group is present in an amount ranging from 10³ U to 10⁵ Upper 100 g of dye composition.

25. The composition according to claim 1, wherein the at least one substrate for the enzyme is an alcohol chosen from primary alcohols, secondary alcohols, long-hydrocarbon-chain alcohols and aromatic alcohols.

26. The composition according to claim 25, wherein the at least one substrate for the enzyme is present in an amount ranging from 0.01% to 60% by weight, relative to the total weight of the composition.

27. The composition according to claim 1, wherein the at least one oxidation dye precursor is an oxidation base chosen from para-phenylenediamines, bis(phenyl)alkylenediamines, para-aminophenols, ortho-aminophenols, heterocyclic bases, and the addition salts thereof.

28. The composition according to claim 27, wherein the at least one oxidation base is present in an amount ranging from 0.0001% to 20% by weight, relative to the total weight of the composition.

29. The composition according to claim 1, wherein the at least one oxidation dye precursor is an oxidation coupler chosen from meta-phenylenediamines, meta-aminophenols, meta-diphenols, naphthalenic couplers, heterocyclic couplers, and the addition salts thereof.

30. The composition according to claim 29, wherein the at least one coupler is present in an amount ranging from 0.0001% to 20% by weight, relative to the total weight of the composition.

31. The composition according to claim 1, further comprising at least one direct dye chosen from cationic and natural direct dyes.

32. A process for dyeing keratin fibers, comprising
applying to the fibers, at least one composition comprising, in a medium that is suitable for dyeing, at least one oxidation dye precursor; at least one alcohol oxidase enzyme; at least one substrate bearing an alcohol functional group for the enzyme and at least one anionic associative polymer,
wherein the substrate can be totally or partially substituted by the oxidation dye precursor when the precursor bears at least one aromatic or aliphatic alcohol functional group, and
leaving the at least one composition on the fibers, for a period of time that is sufficient to develop a desired coloration.

33. The process according to claim 32, wherein the at least one composition is a ready-to-use composition, being stored in anaerobic form, free of oxygen gas, before the time of use.

34. The process according to claim 33, further comprising
separately storing
at least one composition (A) comprising, in a medium that is suitable for dyeing keratin fibers, at least one oxidation dye precursor, and
at least one composition (B) comprising, in a medium that is suitable for keratin fibers, at least one alcohol oxidase enzyme,
wherein the composition (A) and/or the composition (B) comprise at least one substrate for the enzyme, and the composition (A) and/or the composition (B) comprise at least one anionic associative polymer, and
mixing together the compositions (A) and (B) at the time of use before applying this mixture to the keratin fibers.

35. The process according to claim 34, further comprising
separately storing
at least one composition (A) comprising in a medium that is suitable for dyeing keratin fibers, at least one oxidation dye precursor, at least one substrate for an alcohol oxidase enzyme, and at least one anionic associative polymer and
at least one composition (B) comprising, in a medium that is suitable for dyeing keratin fibers, at least one alcohol oxidase enzyme, and
mixing together the compositions (A) and (B) at the time of application, before applying this mixture to the keratin fibers.

36. A multi-compartment dyeing “kit,” comprising
at least one first compartment comprising at least one composition (A) comprising, in a medium that is suitable for dyeing keratin fibers, at least one oxidation dye precursor, and
at least one second compartment comprising at least one composition (B) comprising, in a medium that is suitable for keratin fibers, at least one alcohol oxidase enzyme,
wherein the composition (A) and/or the composition (B) comprise at least one substrate for the enzyme, and the composition (A) and/or the composition (B) comprise at least one anionic associative polymer.
